(19) United States
(12) Patent Application Publication
(10) Pub. No.: US 2025/0152572 A1
(43) Pub. Date: May 15, 2025

(54) NICOTINE FORMULATION

(71) Applicant: INHALER SUPPLIES PTY LTD, Sydney, New South Wales (AU)

(72) Inventors: David Andrew LEWIS, Corsham Wiltshire (GB); Robert David JOHNSON, Corsham Wiltshire (GB); Paul Michael YOUNG, Corsham Wiltshire (GB)

(21) Appl. No.: 18/838,649

(22) PCT Filed: Feb. 17, 2023
(86) PCT No.: PCT/AU2023/050106

§ 371 Date: Aug. 15, 2024

(30) Foreign Application Priority Data
Feb. 18, 2022 (AU) 2022900366

(51) Int. Cl. A61K 31/465; A61K 9/00; A61K 47/10

(52) U.S. Cl. CPC A61K 31/465; A61K 9/008; A61K 47/10

(57) ABSTRACT

This disclosure relates to an improved inhalable formulation comprising nicotine, or a pharmaceutically acceptable derivative or salt thereof, a propellant, a C1 to C6 alcohol and a glycol. The disclosure further relates to a metered dose inhaler containing the inhalable nicotine formulation and a method of treating a nicotine addiction with the formulation.

### TECHNICAL FIELD

This disclosure relates to an inhalable nicotine formulation and a metered dose inhaler containing the inhalable nicotine formulation.

### BACKGROUND

Smoking is a highly addictive habit due, at least in part, to the nicotine contained in tobacco products. Nicotine is a potent stimulant and anxiolytic which gives the smoker a pleasurable feeling. This feeling combined with the habits and rituals of a smoker make it very difficult for the smoker to quit smoking.

Tobacco smoke contains more than 60 cancer-causing chemicals and at least 250 other harmful substances, including hydrogen cyanide, carbon monoxide and ammonia. While e-cigarettes may reduce the number of dangerous chemicals they still rely on heating of a delivery formulation which can result in various harmful aldehydes, ketones and the like being generated and inhaled by the user.

Therefore, it is desirable to provide functional smoking cessation aids. There are various smoking cessation aids currently on the market, such as nicotine skin patches, nicotine-containing gums, nicotine lozenges, nicotine mouth sprays and nicotine inhalators. These aids attempt to deliver nicotine to the subject while avoiding or minimising the release of any of the dangerous side products associated with smoking. However, most of these products only have a limited ability to provide the user with an appropriately satisfying sensation to replace smoking as a primary nicotine resource while reducing the delivery of toxic agents.

In particular, inhaled nicotine products to facilitate smoking cessation may cause harsh sensations on the user's throat due to nicotine deposition in the oropharynx, suboptimal device performance or formulation composition or combinations thereof.

U.S. Pat. No. 9,655,890 describes an inhalable composition containing nicotine, a hydrofluorocarbon propellant, a monodydric alcohol and 0.1 to 1% of a glycol and/or glycol ether, wherein the ratio of monohydric alcohol:glycol or glycol ether by weight is from 3:1 to 1:1. An unmetered dose of the nicotine composition can be delivered orally through a pressurised container, such as a simulated cigarette. However, during their clinical study, seventeen of the 59 participants (29%) reported throat irritation. There is an ongoing need to provide an inhalable formulation that minimises throat irritation.

WO 2015/128599 A1 describes a semi-continuous process for preparing a formulation comprising nicotine and a propellant, and optionally propylene glycol and ethanol. The application focuses on providing a process to make a nicotine-containing formulation and does not discuss if the prepared formulation can be delivered to the deep lungs of the subject.

Metered dose inhalers (MDIs) are used to treat respiratory diseases by delivering a reliable, consistent dose of a pharmaceutical to the patients' airways through inhalation. They do not rely on heating and are safe and convenient for users to carry and draw an inhalation breath from when in use. However, MDIs present challenges in terms of suitable formulations which will generate appropriate particle sizes containing the active agent. Since they do not rely on heating, and produce almost instantaneous evaporation of multiple formulation components, it is necessary to achieve a thermodynamic balance of the formulation components to ensure the active agent is appropriately maintained within the droplets so that the active agent can be delivered to the lungs.

This is particularly challenging when the active agent is a highly volatile compound such as nicotine. The formulation needs to be such that in being dispensed from an MDI the volatile nicotine is restrained from quickly evaporating and separating from other components of the formulation and depositing in the oral cavity or pharynx. This requires control of a highly dynamic system of formulation components which are expanding, following release from the MDI, and rapidly cooling and condensing. Careful balance of the relative solubilities of the components is crucial along with achieving particles having an appropriate average particle or droplet diameter to provide desirable delivery to the deep lungs.

### SUMMARY

In a first aspect, the disclosure resides in an inhalable formulation comprising:
nicotine, or a pharmaceutically acceptable derivative or salt thereof;
at least 90% w/w of a propellant;
between 2%-8% w/w of a C₁ to C₆ alcohol; and
between 1.5%-5% w/w of a glycol.

In a second aspect, the disclosure resides in a metered dose inhaler comprising an inhalable formulation, said inhalable formulation comprising:
nicotine, or a pharmaceutically acceptable derivative or salt thereof;
at least 90% w/w of a propellant;
between 2%-8% w/w of a C₁ to C₆ alcohol; and
between 1.5%-5% w/w of a glycol.

In embodiments, the nicotine, or a pharmaceutically acceptable derivative or salt thereof, is present at between 200 μg/50 μL w/v to 50 μg/50 μL w/v of the entire formulation.

In embodiments, the nicotine, or a pharmaceutically acceptable derivative or salt thereof, is present at between about 150 μg/50 μL w/v to about 75 μg/50 μL w/v of the entire formulation.

In embodiments, the propellant is a hydrofluorocarbon propellant.

In embodiments, the hydrofluorocarbon propellant is selected from the group consisting of HFA 134a, HFA 152a, HFA 227 and HFO 1234ze.

In embodiments, the C₁ to C₆ alcohol is selected from a C₁ to C₄ alcohol, a C₂ to C₄ alcohol, and a C₂ or C₃ alcohol.

In a preferred embodiment, the C₁ to C₆ alcohol is ethanol.

In embodiments, the C₁ to C₆ alcohol is present at between 2%-8% w/w of the entire inhalable formulation.

In another embodiment, the C₁ to C₆ alcohol is present at between 3%-7% w/w of the entire inhalable formulation.

In another embodiment, the C₁ to C₆ alcohol is present at about 5% w/w of the entire inhalable formulation.

In a preferred embodiment, the glycol is propylene glycol.

In an embodiment, the glycol content is between 1.5%-5% w/w of the entire inhalable formulation.

In another embodiment, the glycol content is between 2%-4% w/w of the entire inhalable formulation.

In embodiments, the inhalable formulation may further comprise glycerol.

In an embodiment, the glycerol content is between 0.01%-0.5% w/w of the entire inhalable formulation.

In another embodiment, the glycerol content is between 0.05%-0.25% w/w of the entire inhalable formulation.

In another embodiment, the glycerol content is around 0.1% w/w of the entire inhalable formulation.

In a third aspect, the disclosure resides in a method of delivering nicotine to a subject including the steps of:
providing an inhalable formulation of the first aspect to the subject;
allowing the subject to inhale the inhalable formulation,
to thereby deliver the nicotine to the subject.

In embodiments of the third aspect, the method of delivering nicotine is a method of delivering nicotine to the lungs of the subject.

In a fourth aspect, the disclosure resides in a method of treating a nicotine addiction in a subject including the steps of:
providing a first inhalable formulation of the first aspect to the subject;
allowing the subject to inhale the formulation,
to deliver nicotine to the subject and thereby treat the nicotine addiction.

In embodiments of the fourth aspect, the method of treating a nicotine addiction in a subject further includes a step of administering to the subject one or more further inhalable nicotine formulations having a reduced nicotine content as compared with the inhalable formulation previously administered.

Each aspect or embodiment as defined herein may be combined with any other aspect(s) or embodiment(s) unless clearly indicated otherwise.

### BRIEF DESCRIPTION OF DRAWINGS

FIG. 1. shows a Dosage Unit Sampling Apparatus (DUSA) for the evaluation of particle-vapour phase nicotine mass. The setup consists of a vapour trap, a DUSA/filter and an actuator. The flow rate is around 30 L per minute.

FIG. 2. shows the apparatus for evaluation of particle size distribution.

FIG. 3. shows the nicotine deposition within the DUSA and vapour trap apparatus.

FIG. 4. shows the particle size distribution (%) for an inhalable formulation comprising 3% w/w ethanol, 2% w/w PG in HFA 134a (50 μg/50 μL nicotine).

FIG. 5. shows the particle size distribution (μg) for an inhalable formulation comprising 3% w/w ethanol, 2% w/w PG in HFA 134a (50 μg/50 μL nicotine).

FIG. 6. shows that the formulations prepared with HFA 152a (left) and HFO1234ze (right) are clear solutions at 5° C.

### DESCRIPTION OF EMBODIMENTS

The high volatility of nicotine creates great challenges when being used in an MDI formulation since the compound has a natural tendency to evaporate quickly and to separate from the other components in the formulation, thereby leading to it being considerably more likely to settle in the oral cavity rather than travelling to the deep lungs for appropriate effect. The unique and challenging physical and chemical properties of nicotine cannot easily be compared to other actives, such as cannabinoids, which can be found used in inhalable formulations and requires a bespoke formulation approach.

In contrast to nicotine, which is a liquid, cannabinoids, such as CBD and THC, are solid powders at room temperature. If left open to the atmosphere, CBD and THC will remain a solid powder while nicotine will eventually vaporise and evaporate. An evaporating aerosol from an MDI containing either CBD or THC will result in residual non-volatile CBD or THC droplets, respectively.

CBD and THC behave very much like typical inhalable pharmaceutical drugs. For example, drug delivery measurements can be performed at room temperature and the residual (non-volatile) drugs can be collected upon filters and cascade impactors. Both CBD and THC can be delivered to the deep lungs by an MDI using formulations simply containing ethanol and propellant alone due to their physical properties.

Nicotine, on the other hand, is very different and considerably more challenging. When the MDI is pressed, a puff of formulation containing nicotine is emitted from the MDI. However, nicotine is highly volatile (particularly in aerosol format), making it extremely difficult to quantify using USP techniques. A residual nicotine droplet will not be formed; instead a vapor will be created. Dilution of the expanding aerosol by the surrounding air stream will occur rapidly, and this will drive the nicotine from the aerosol droplets into the surrounding air. The result is that inhaled nicotine vapor will be lost to the surfaces of the oropharynx; resulting in harsh sensation by the patient and failure to deliver nicotine to the lungs. This therefore presents a very significant challenge to control this process appropriately to achieve efficient delivery into the deep lungs.

It is in this context that it is particularly surprising that the volatility of a nicotine dose delivered from a metered dose inhaler can be controlled by careful selection of formulation excipients and composition. The present disclosure describes how an inhalable nicotine formulation can be uniquely tailored for delivery via a MDI to a subject's lungs to facilitate smoking cessation.

In both conventional and electronic cigarettes, nicotine must be heated in order to be delivered to the lungs of the subject via inhalation. The heating process can result in the formation of harmful by-products, such as aldehydes, ketones, nitrosamines and heavy metals, which are also delivered to the subject. In contrast, the present disclosure provides an inhalable nicotine formulation that can be delivered without the need for heating. Furthermore, the present disclosure provides for a MDI containing the inhalable formulation that can deliver the formulation via inhalation deep to the lungs of a subject without heating.

By carefully maintaining control of the nicotine volatility, by selection of formulation components and relative amounts, it has advantageously been found that aerosol particles or droplets emitted by a MDI can retard the nicotine from separating from the particle too early in the inhalation process and thereby be deposited within the deep lung rather than the oral cavity or pharynx and so greatly improve user sensation/experience as well as maximise delivery of the available nicotine.

Particularly, the present disclosure describes the use of a C₁ to C₆ alcohol, particularly ethanol, and a glycol, particularly propylene glycol, to form a particle encapsulating the volatile nicotine. These components, in certain % w/w amounts in an inhalable formulation, can appropriately control the diffusion of nicotine to the particle surface and its subsequent evaporation. This control occurs in spite of the complex thermodynamic changes occurring in the particle relating to expansion of the formulation, the rapid flash evaporation of the hydrofluorocarbon propellant and the consequent cooling and the ongoing partitioning of the alcohol, glycol and nicotine while they too seek to escape and evaporate from the particle.

Without wishing to be bound by theory, particles within liquids generally follow a diffusion gradient from high to low concentrations and, therefore, move to the particle surface to exit the liquid droplet. Likewise, the diffusion of nicotine from the surface of the aerosol particles is proportional to the concentration gradient of nicotine at the surface of the particle and just above the surface of the particle. Thus, by careful design of the inhalable formulation, the processes that occur during droplet formation and aerosol maturation phases has been engineered such that propellant flashing and partial ethanol evaporation occurs and controlled nicotine partitioning also takes place within remaining formulation components. The nicotine is encapsulated within the particle and its diffusion to the particle surface is hindered and evaporation into the surrounding airstream is prevented which allows for optimal delivery of the nicotine to the lungs of the subject.

In a first aspect, the disclosure resides in an inhalable formulation comprising
nicotine, or a pharmaceutically acceptable derivative or salt thereof;
at least 90% w/w of a propellant;
between 2%-8% w/w of a C₁ to C₆ alcohol; and
between 1.5%-5% w/w of a glycol.

In embodiments, the nicotine, or a pharmaceutically acceptable derivative or salt thereof, is present in the inhalable formulation at between about 200 μg/50 μL w/v to about 50 μg/50 μL w/v, or 190 μg/50 μL w/v to about 55 μg/50 μL w/v, or 180 μg/50 μL w/v to about 60 μg/50 μL w/v, or 170 μg/50 μL w/v to about 65 μg/50 μL w/v, or 160 μg/50 μL w/v to about 70 μg/50 μL w/v.

In embodiments, the nicotine, or pharmaceutically acceptable salt thereof, is present in the inhalable formulation at between about 150 μg/50 μL w/v to about 70 μg/50 μL, or w/v 140 μg/50 μL w/v to about 70 μg/50 μL w/v, or 130 μg/50 μL w/v to about 70 μg/50 μL w/v, or 120 μg/50 μL w/v to about 70 μg/50 μL w/v, or 110 μg/50 μL w/v to about 70 μg/50 μL w/v.

In an embodiment, the nicotine, or pharmaceutically acceptable salt thereof, is present in the inhalable formulation at between about 100 μg/50 μL w/v to about 75 μg/50 μL w/v.

In embodiments, it may be desired to provide alternative inhalable formulations each with decreasing nicotine content to aid with weaning a subject off a nicotine addiction.

In embodiments, the nicotine, or a pharmaceutically acceptable derivative or salt thereof, is present in the inhalable formulation at about 150 μg/50 μL w/v, or about 125 μg/50 μL w/v or about 100 μg/50 μL w/v or about 75 μg/50 μL w/v or about 50 μg/50 μL w/v.

A range of pharmaceutically acceptable nicotine derivatives or salts are known in the art and their use with the present inhalable formulation is not particularly limited. Nicotine is an alkaloid that can be isolated as a free-base, but, when combined with an acid, it becomes protonated and forms a salt. For the purposes of the present disclosure the nicotine may be used as the free base or it may be used in form of an acid salt formed with an acid. Suitable acids to form a salt of nicotine may be selected from those which are known in art of formulation for use in ingestible, particularly inhalable, formulations for human consumption. In certain embodiments, the nicotine salt may be formed using an acid selected from the group consisting of formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, tartaric acid, bitartaric acid, lactic acid, malonic acid, succinic acid, fumaric acid, finnaric acid, gluconic acid, saccharic acid, malonic acid, and malic acid. In preferred embodiments, the suitable organic acids may be selected from, but are not limited to, glycolic, pyruvic, lactic, levulinic, fumaric, succinic, benzoic, salicylic, malic, tartaric, and citric acids.

In embodiments wherein the nicotine is present in the inhalable formulation as a salt form it may be present as one or more of a lactic, benzoic or levulinic acid salt.

In embodiments wherein the nicotine is present in the inhalable formulation as a derivative it may be present as one or more of an N-oxide derivative, a glucuronide derivative, an N-alkyl derivative or an isomer of nicotine or any such derivatives. In embodiments, the propellant is a hydrofluorocarbon propellant.

In embodiments, the hydrofluorocarbon propellant is a hydrofluoroalkane or hydrofluoroalkene.

In an embodiment, the hydrofluoroalkane or hydrofluoroalkene propellant is selected from the group consisting of 1,1,2,2-tetrafluoroethane (HFA 134a), 1,1-difluoroethane (HFA 152a), 1,1,1,2,3,3-heptafluoropropane (HFA 227) and trans-1,3,3,3-tetrafluoroprop-1-ene (HFO 1234ze). These hydrofluoroalkanes and hydrofluoroalkenes are particularly effective as propellants and have no adverse effect on the human body.

In a preferred embodiment, the propellant is selected from the group consisting of HFA 134a, HFA 152a and HFO 1234ze.

In embodiments, the propellant is present in at least 90% w/w of the entire formulation, or at least 92% w/w of the entire formulation, or at least 94% w/w of the entire formulation, or at least 95% w/w of the entire formulation.

In embodiments, the C₁ to C₆ alcohol is selected from a C₁ to C₄ alcohol, a C₂ to C₄ alcohol, and a C₂ or C₃ alcohol.

In embodiments, the C₁ to C₆ alcohol is selected from methanol, ethanol, propanol and isopropanol.

In a preferred embodiment, the C₁ to C₆ alcohol is ethanol. The use and amount of ethanol may provide benefits for the solubility of the nicotine in the formulation. It has been found that particular levels of ethanol and glycol create an ideal balance in the solubility and partitioning, within the particle, of each component and appropriately retain the nicotine on this basis within the particle. Decreasing the ethanol content below certain levels reduces the solubility profile of the particle such that further undesirable changes have to be made in the levels of other components of the formulation to compensate.

In embodiments, the C₁ to C₆ alcohol content is between 2%-8% w/w, or between 2%-7% w/w, or between 2%-6% w/w, or between 2%-5% w/w, or between 3%-8% w/w, or between 3%-7% w/w, or between 3%-6% w/w, or between 3%-5% w/w, or between 4%-8% w/w, or between 4%-7% w/w, or between 4%-6% w/w, or between 5%-7% w/w, of the entire formulation.

In embodiments, the C₁ to C₆ alcohol content is about 6% w/w, or about 5.5% w/w, or about 5% w/w, or about 4.5% w/w, or about 4% w/w of the entire formulation.

In embodiments, the glycol is selected from the group consisting of propylene glycol, polypropylene glycol and polyethylene glycol.

In a preferred embodiment, the glycol is propylene glycol.

It has been found that a certain level of propylene glycol, on a % w/w basis, provides for encapsulation of the nicotine following expulsion from the MDI. This is essential to avoid the nicotine simply flashing out of the formulation along with the propellant and some of the ethanol immediately after activation of the MDI. It is postulated that these levels of propylene glycol retard the movement of the nicotine towards the outer surface of the formed particle from which it could escape prior to delivery to the subject's lungs. The relative amounts of ethanol to propylene glycol are important in achieving this appropriate solubility and release profile for the nicotine as well as providing for a desirable particle size.

In embodiments, the glycol content is between 1.5%-5.0% w/w, or between 2.0%-5.0% w/w, or between 2.5%-5.0% w/w, or between 3.0%-5.0% w/w, or between 1.5%-4.5% w/w, or between 2.0%-4.5% w/w, or between 2.5%-4.5% w/w, or between 1.5%-4.0% w/w, or between 2.0%-4.0% w/w, or between 2.5%-4.0% w/w, or between 3.0%-5.0% w/w, of the entire formulation.

In embodiments, the glycol content is present in the inhalable formulation at about 4.0% w/w, about 3.5% w/w, about 3.0% w/w, about 2.5% w/w, or about 2.0% w/w of the entire formulation.

In some embodiments, it may be beneficial to further add glycerol to the inhalable formulation to modify the properties thereof. In another embodiment, the formulation does not contain any glycerol, and it is an advantage of the present invention that glycerol is not an essential component for effective nicotine delivery.

In embodiments, the glycerol is present at between 0.01%-0.5% w/w, between 0.05%-0.25% w/w, or between 0.075%-0.2% w/w, of the entire formulation.

In another embodiment, the glycerol is present at about 0.1% w/w of the entire formulation.

The careful balance of all components in the formulation is important for the invention to provide the observed benefits of delivering the nicotine deep to the lungs. As demonstrated in the Examples, if the ethanol content is too low or no propylene glycol was added to the formulation, most of the nicotine was trapped in the device instead of being delivered to the subject. This is representative of nicotine escaping from the particle within the oral cavity or pharynx of the user and does not provide for delivery to the lungs.

In embodiments, the inhalable formulation further comprises a flavour-masking agent. Flavour-masking agents refer to a variety of flavour materials of natural or synthetic origin. They include single compounds and mixtures and are commonly employed in formulating ingestible compositions to improve acceptance of the user. Preferably, the flavour-masking agent has flavour properties that enhance the user's sensory experience of the inhalable formulation.

Suitable flavours and aromas include, but are not limited to, any one or more natural or synthetic flavour or aroma, such as tobacco, smoke, chocolate, liquorice, citrus and other fruit flavours, 1-menthol, gamma octalactone, vanillin, ethyl vanillin, breath freshener flavours, spice flavours such as cinnamon, methyl salicylate, linalool, bergamot oil, geranium oil, lemon oil, and ginger oil, and the like.

Other suitable flavours and aromas may include flavour compounds selected from the group consisting of an acid, an alcohol, an ester, an aldehyde, a ketone, a pyrazine, combinations or blends thereof and the like. Suitable flavour compounds may be selected, for example, from the group consisting of phenylacetic acid, solanone, megastigmatrienone, 2-heptanone, benzylalcohol, cis-3-hexenyl acetate, valeric acid, valeric aldehyde, ester, terpene, sesquiterpene, nootkatone, maltol, damascenone, pyrazine, lactone, anethole, iso-s valeric acid, combinations thereof, and the like.

In embodiments, the inhalable formulation does not comprise an aroma and/or flavour-enhancing oil. In embodiments, the inhalable formulation does not comprise a menthol-containing essential oil or peppermint oil.

The size of the particles in the inhalable formulation plays an important role to achieve the desired delivery to the deep lungs. In embodiments, the particle size is less than 10 μm, or less than 5 μm. It may be beneficial to have an even smaller particle size of between 0.5 μm to 3.5 μm or between 0.5 μm to 2 μm to allow the particles to reach the deep lung. In a preferred embodiment, the particle size is between 0.5 μm to 2 μm or between 0.5 μm to 1 μm.

In a second aspect, the disclosure resides in a metered dose inhaler comprising an inhalable formulation, said inhalable formulation comprising:
nicotine, or a pharmaceutically acceptable derivative or salt thereof;
at least 90% w/w of a propellant;
between 2%-8% w/w of a C₁ to C₆ alcohol; and
between 1.5%-5% w/w of a glycol.

Metered dose inhalers are well-known in the art and a wide range of such MDIs may be selected for use with the formulation of the first aspect. Such MDIs are familiar to the person of skill in the art and may be selected from those which are commercially available. Particularly suitable MDIs will be any designed for use in the delivery of volatile compounds.

The MDIs which are available all have a container which can be attached to the MDI or which may be built into it and which can contain the active agent being delivered. Such containers are adapted to contain a pressurised fluid and often are designed for containing hydrofluorocarbon-type propellant formulations.

The diameter of the aperture of the MDI may influence the average particle size which is subsequently formed and so can have an influence on delivery.

In embodiments, the aperture of the MDI has a diameter of between 0.10 mm and 0.50 mm, or between 0.15 mm and 0.40 mm or between 0.20 mm and 0.35 mm. In an embodiment, the aperture has a diameter of about 0.22 mm or about 0.30 mm.

In a third aspect, the disclosure resides in a method of delivering nicotine to a subject including the steps of:
providing an inhalable formulation of the first aspect to the subject;
allowing the subject to inhale the inhalable formulation,
to thereby deliver the nicotine to the subject.

In embodiments of the third aspect, the method of delivering nicotine is a method of delivering nicotine to the lungs of the subject.

The careful design and balance of all components of the inhalable formulation of the present disclosure mean that, during the droplet formation and aerosol maturation process, the nicotine is encapsulated within the fluid particle. Diffusion of the nicotine to the particle surface is hindered by the balance of ethanol, propylene glycol and, in preferred embodiments, glycerol, and evaporation into the surrounding airstream is retarded and, consequently, the nicotine can be delivered to the deep lungs of the subject.

This is a distinct advantage of the formulation of the present disclosure. Most prior art approaches either use significantly more volatile fluid components or employ relevant amounts of excipients which cannot achieve a balance between solubility of the nicotine and retention of the nicotine within the fluid droplets following expulsion from the MDI. Most known approaches simply ignore the complex thermodynamics of this multi-component system and result in rapid deposition of the nicotine within the oral cavity or pharynx of the user thereby resulting in a harsh sensation in the pharynx and a failure to properly deliver the nicotine to the lungs.

In a fourth aspect, the disclosure resides in a method of treating a nicotine addiction in a subject including the steps of:
providing a first inhalable formulation of the first aspect to the subject;
allowing the subject to inhale the formulation,
to deliver nicotine to the subject and thereby treat the nicotine addiction.

In embodiments of the method, the method of treating a nicotine addiction in a subject further includes a step of administering a second inhalable formulation of the first aspect, the second inhalable formulation having a reduced nicotine content compared with the first inhalable formulation.

The method of the fourth aspect may include the inhalation of third, fourth, fifth and further inhalable formulations of the first aspect, as required, with each subsequent inhalable formulation having a reduced nicotine content compared with the previously administered inhalable formulation.

It will be appreciated that the first, second, third, fourth and further inhalable formulations may be administered or inhaled by the subject multiple times throughout a time period before it is appropriate to move to the next inhalable formulation having reduced nicotine content. In this manner, over the course of the treatment regimen, the effect is to wean the subject off the nicotine addiction.

In embodiments of the method, the treatment may include 1, 2, 3, 4, 5, 6 or 7 additional steps of administering a nicotine formulation with reduced nicotine content depending on the severity of the person's addiction.

In embodiments, any one inhalable nicotine formulation may be used for 1, 2 or 3 weeks before further reducing the nicotine content. It may be beneficial to reduce the nicotine content in 25 μg/50 μL or 50 μg/50 μL increments with other aspects of the inhalable formulations being kept the same.

The length of the treatment course varies depending on the severity of the subject's addiction. In embodiments, the length of the treatment course may be 4, 5, 6, 7, 8, 9, 10, 11 or 12 weeks.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXPERIMENTAL

### Materials:

15 ml glass Aerosol tubes, St Gobain, Batch 711175
14 ml C0842 plasma coated canisters, Presspart, Batch PPB0021336 and 19 ml plasma treated canisters, Presspart, Batch PPB0022230
50 μl Metering Valves for inverted use (EPDM gaskets EF327), Bespak and Aptar
Continuous spray valve for glass bottle inverted use, Bespak, Batch BK0642138
0.30 mm BK634™ Actuators, Bespak, Batch BK0486487
0.22 mm BK633™ Actuators, Bespak, Batch BK0130974
0.23 mm Actuators, Presspart, Batch PPT0031512
Nicotine, Sigma, Batch S7840677047
Glycerol, Sigma, Batch BCBK0214V
Propylene Glycol, Batch SHBM3251
Ethanol, SLS, Batch 315327, and Sigma, Batch SHBL0003
Lactic Acid 90%, Sigma, Batch BCCF1169.
HFA 134a, Mexichem, Batch RB21634-1, and Zephex MDI, Mexichem
HFA 152a, Mexichem
HFO 1234ze, Honeywell

### Methodology:

Metered dose inhalers (50 μg/50 μl Nicotine dose) were manufactured by weight using a two-stage fill methodology. Predetermined quantities of Nicotine, Ethanol, Propylene Glycol, Glycerol and Lactic acid were weighed into a glass stoppered conical flask and mixed to form clear bulk solutions. Bulks were weighed into either 15 ml St Gobain glass bottle aerosol tubes or Presspart 14 ml C0842 plasma coated canisters; crimped with Bespak valves; and gassed with HFA 134a propellant using a Pamasol P2016 laboratory filling plant.

Formulations packaged in glass bottles were used to confirm the final formulations were clear solutions at T=5° C. and T=20° C.

Formulations packaged in 14 ml canisters (Presspart) and 50 μl valves (Bespak) were used to characterise nicotine delivery. All MDIs were actuated using BK630 series Actuators (Bespak).

Table 1 presents the formulations investigated. Batch OZ211018/DS/A (see Table 1) was based upon earlier known formulations provided by the client. However, a 50 μl (rather than 75 μl) metering valve was utilised; thus, nicotine and lactic acid concentrations were adjusted accordingly.

TABLE 1
Formulations investigated

| | | Nicotine/ | | Propylene | | |
|---|---|---|---|---|---|---|
| | Nicotine | Lactic Acid | Ethanol | Glycol | Glycerol | HFA |
| Batch | μg/50 μl | 90% | (% w/w) | %(w/w) | %(w/w) | 134a |
| OZ211018/ | 50 | 2.1 ± 0.1 | 5.0 | 0.0 | 0.0 | to |
| DS/L | | | | | | 100% w/w |
| OZ211018/ | | | 8.0 | 0.0 | 0.5 | |
| DS/M | | | | | | |
| OZ211018/ | | | 0.2 | 0.0 | 0.0 | |
| DS/A | | | | | | |
| OZ211018/ | | | 8.0 | 2.0 | 0.5 | |
| DS/N | | | | | | |
| OZ211018/ | | | 8.0 | 3.0 | 0.1 | |
| DS/O | | | | | | |
| OZ211018/ | | | 5.0 | 2.0 | 0.1 | |
| DS/Q | | | | | | |
| OZ211018/ | | | 4.9 | 3.0 | 0.0 | |
| DS/G | | | | | | |
| OZ211018/ | | | 5.0 | 3.0 | 0.1 | |
| DS/H | | | | | | |
| OZ211018/ | | | 5.1 | 4.1 | 0.1 | |
| DS/P | | | | | | |

Volatility of formulations were evaluated by use of a Dosage Unit Sampling Apparatus (DUSA), USP methodology with the addition of a Drechsel Gas Trap containing 70 ml of diluent, see FIG. 1.

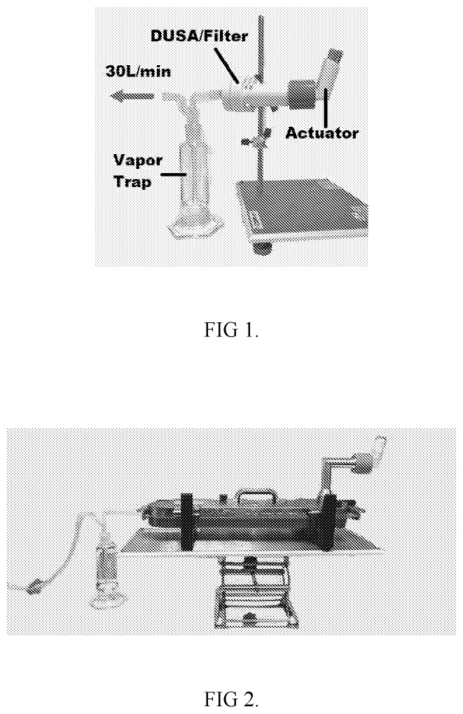

In accordance with USP <601>, particle size distributions were evaluated by next generation impactor fitted with USP induction port (NGI, Copley Scientific Limited) (see FIG. 2). The NGI was chilled for 2 hours at 5° C. before sampling two doses from each test inhaler at flow rate of 30 L/min.

For each test inhaler the drug deposition within each stage of the NGI was determined by quantitative extraction to volume with methanol and determination of nicotine within each test sample by a high pressure liquid chromatography (HPLC). Drug delivery metrics and particle size distribution was calculated using CITDAS V3.10 software (Copley Scientific Limited).

### Example 1: Results for Investigated Formulations

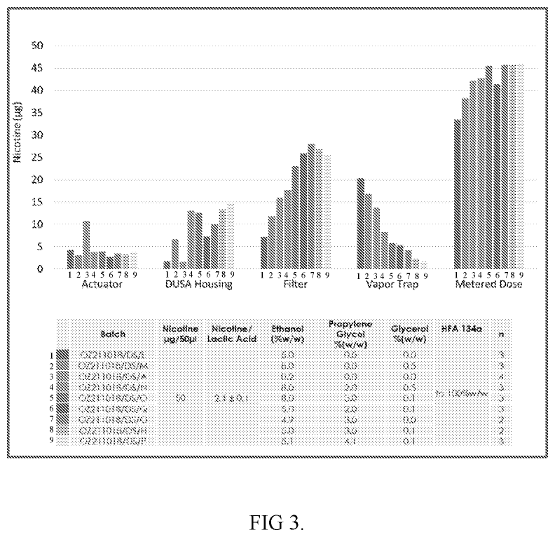

Data obtained for the MDI Batches evaluated with the DUSA/Trap Apparatus are presented in Table 2 and FIG. 3.

TABLE 2
Results: DUSA/trap nicotine mass observed
(in order of decreasing vapour trap mass)

| | | | | Metered | |
|---|---|---|---|---|---|
| | Nicotine Deposition per Dose (μg) | | | Nicotine | Shot |
| | | DUSA/ | | Dose | Weight |
| Batch | Actuator | Filter | Trap | (μg) | (mg) |
| OZ211018/ | 4.2 ± | 8.8 ± | 20.3 ± | 33.4 ± | 56.5 ± |
| DS/L | 0.3 | 1.9 | 2.8 | 1.0 | 0.7 |
| OZ211018/ | 3.1 ± | 18.4 ± | 16.8 ± | 38.2 ± | 58.3 ± |
| DS/M | 0.4 | 2.0 | 1.5 | 0.2 | 0.1 |
| OZ211018/ | 11.5 ± | 17.9 ± | 14.7 ± | 44.2 ± | 63.3 ± |
| DS/A | 1.6 | 2.7 | 4.0 | 4.5 | 0.8 |
| OZ211018/ | 3.8 ± | 30.7 ± | 8.3 ± | 42.7 ± | 58.9 ± |
| DS/N | 0.6 | 3.8 | 3.5 | 1.1 | 0.3 |
| OZ211018/ | 3.9 ± | 35.7 ± | 5.9 ± | 45.5 ± | 59.1 ± |
| DS/O | 0.3 | 5.6 | 4.4 | 1.0 | 0.2 |
| OZ211018/ | 2.7 ± | 33.2 ± | 5.4 ± | 41.4 ± | 58.4 ± |
| DS/Q | 0.5 | 1.4 | 0.4 | 1.5 | 1.0 |
| OZ211018/ | 3.5 ± | 38.0 ± | 4.2 ± | 45.7 ± | 60.1 ± |
| DS/G | 0.0 | 0.9 | 0.7 | 0.2 | 0.4 |
| OZ211018/ | 3.3 ± | 40.1 ± | 2.2 ± | 45.7 ± | 59.9 ± |
| DS/H | 0.7 | 0.8 | 0.7 | 0.6 | 0.6 |
| OZ211018/ | 3.8 ± | 40.2 ± | 1.9 ± | 45.9 ± | 60.3 ± |
| DS/P | 0.4 | 0.4 | 0.3 | 0.3 | 0.2 |

Except for the very low ethanol formulation (0.2% w/w ethanol), actuator nicotine deposition was similar for all batches (range: 3-4 μg; <10% of the target 50 μg metered dose). The low ethanol content formulation (batch OZ211018/DS/A) had an actuator nicotine deposition of 12±2 μg (24% of the 50 μg target dose).

Metered dose values approached 46 μg (92% of the 50 μg target dose) as the vapour mass measured in the trap decreased. Metered dose was lowest (33 μg, 66% of the target 50 μg metered dose) for the formulation with the most nicotine measured within the vapour trap.

The formulation composition had a key impact upon whether the nicotine was efficiently collected within the DUSA/Filter (particulate) or was passed through to be collected in the vapour trap.

High nicotine was observed in the vapour trap for formulations not containing low/non-volatile excipients (propylene glycol (PG) and glycerol (VG) respectively).

Low nicotine was observed in the vapour trap for formulations that did contain low/non-volatile excipients (propylene glycol (PG) and glycerol (VG) respectively).

However, formulation batch OZ211018/DS/M indicates that 8% w/w ethanol 0.5% w/w VG is not enough to encapsulate the nicotine. Addition of small quantities of PG were required to reduce vapour trap nicotine mass. Furthermore, reduction of ethanol from 8% w/w to 5% w/w further decreased the mass of nicotine in the vapour trap.

The experiments presented demonstrate an important interplay between the solvent components and indicate that preferred combinations contain approximately 5% w/w ethanol which appears to be an optimal amount to solubilize PG when present within a 2-4% w/w range with an optional but advantageous small addition of VG at approximately 0.1% w/w.

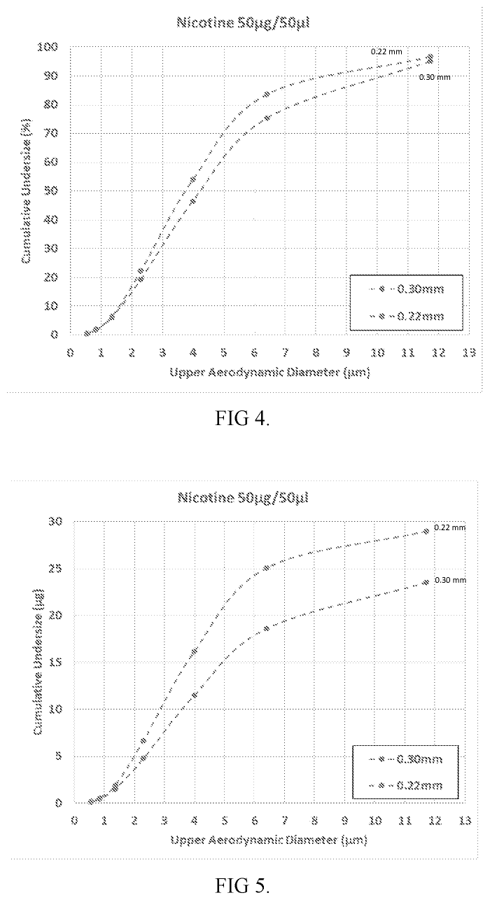

The particle size distribution data for an additional 50 μg/50 μl Nicotine, 3% w/w ethanol, 2% w/w PG formulation in HFA 134a for actuator orifice diameters 0.22 mm and 0.30 mm is shown in FIG. 4 and FIG. 5. A fine particle dose <5 μm of 22 μg and 15 μg was observed for each respective actuator. A further reduction in PG towards 1% w/w would reduce the mass median aerodynamic diameter (MMAD) from the rather high value of 4.2 μm (0.30 mm) and 3.7 μm (0.22 mm) to approximately 3 μm. It is interesting to note that no nicotine was observed in the vapour trap for either of these impactor measurements and metered dose values were 44 μg and 42 μg respectively. When evaluated with the DUSA/Filter apparatus a metered dose of 43 μg was observed with just 4 μg in the vapour trap.

### Example 2: Reduction of Propylene Glycol (PG) Content to 1% w/w in 3% w/w Ethanol

In an additional evaluation, PG content was reduced to 1% w/w. It was necessary to include 3% w/w ethanol content to maintain miscibility of the formulation. Glycerol was also included at a level of 0.09% w/w. The observed drug delivery data is presented in Table 3. Three canisters of the batch were evaluated in duplicate by Next Generation Impactor (NGI; n=6). Metered dose and delivered dose were close to target 51±1 μg and 47±1 μg respectively. Fine particle dose <5 μm aerodynamic diameter was 28±2 μg (FPF=60±5%) and MMAD reduced to 3.4±0.2 μm.

TABLE 3
Drug Delivery observed by NGI for 50 μg/50 μl nicotine,
0.4% w/w lactic acid, 3% w/w ethanol, 0.09% w/w glycerol
in HFA 134a (Batch: OZ211122/DAL/A, Cans 1, 2 & 3).

| | |
|---|---|
| Metered Dose (μg) | 51.1 ± 1.1 |
| Delivered Dose (μg) | 46.6 ± 1.0 |
| Fine Particle Dose (μg) | 27.8 ± 2.0 |
| Fine Particle Fraction (%) | 59.6 ± 4.6 |
| MMAD (μm) | 3.4 ± 0.2 |
| n | 6 |

However, reducing the PG content also effected the ability for nicotine encapsulation. When evaluated by DUSA with vapour trap, 13.7±7.0 μg (out of a total of 47.7±3.1 μg) reached the trap. This would rank as the fourth most volatile formulation described in this patent (according to the data presented in Table 2 and FIG. 1), suggesting that 2-4% w/w PG is required within low ethanol content (e.g. 5% w/w) formulations to achieve a low volatile “encapsulated” nicotine formulation. Reducing PG or increasing ethanol content has been observed to increase nicotine volatility.

TABLE 4
Drug Delivery observed by DUSA/Vapour Trap for 50 μg/50
μl nicotine, 0.4% w/w lactic acid, 3% w/w ethanol, 0.09%
w/w glycerol in HFA 134a (Batch: OZ211122/DAL/A, Cans 1, 2 & 3).

| | |
|---|---|
| Metered Dose (μg) | 47.7 ± 3.1 |
| Delivered Dose (μg) | 43.5 ± 3.2 |
| Actuator (μg) | 4.2 ± 0.8 |
| DUSA (μg) | 3.8 ± 1.4 |
| Filter (μg) | 26.0 ± 9.8 |
| Trap (μg) | 13.7 ± 7.0 |
| n | 6 |

In conclusion, it is possible to control the volatility of nicotine delivered from a metered dose inhaler. Inclusion of propylene glycol (PG), glycerol (VG) and ethanol at low levels has demonstrated that nicotine delivery approaches the performance of a non-volatile molecule. However, when reducing PG to 1% w/w and ethanol to 3% w/w the volatility of nicotine was “restored”; demonstrating that careful balancing of formulation content is required if a low volatile nicotine formulation is to be achieved.

### Example 3: Formulation with HFA 134a

The following table shows a formulation comprising nicotine, lactic acid, ethanol, propylene glycol and propellant HFA 134a.

TABLE 5
Formulation for MDI nicotine.

| | | | Mass per 50 μL |
|---|---|---|---|
| Component | Mass % w/w | Per pMDI (mg) | (mg) |
| Nicotine | 0.0843 | 14.007 | 0.050 |
| Ethanol | 5.0000 | 830.813 | 2.967 |
| Propylene Glycol | 3.0000 | 498.488 | 1.780 |
| Menthol | 0.0000 | 0.000 | 0.000 |
| Glycerol | 0.0000 | 0.000 | 0.000 |
| Lactic Acid | 0.0562 | 9.338 | 0.033 |
| HFA 134a | 91.8595 | 15263.604 | 54.513 |
| Total | 100.0000 | 16616.250 | 59.344 |

### Example 4: Alternative Propellants in MDI Nicotine Formulation

In the following experiments, the propellant HFA 134a has been replaced with HFA 152a and HFO 1234ze and particle size distribution was measured.

Metered dose inhalers (50 μg/50 μl Nicotine dose) were manufactured by weight using a two-stage fill methodology. Pre-determined quantities of Nicotine, Ethanol, Propylene Glycol, and Lactic acid were weighed into a glass stoppered conical flask and mixed to form clear bulk solutions. Bulks were weighed into either 15 ml St Gobain glass bottle aerosol tubes or Presspart 19 ml C0128 plasma treated canisters; crimped with Aptar valves; and gassed with propellant using a Pamasol P2016 laboratory filling plant.

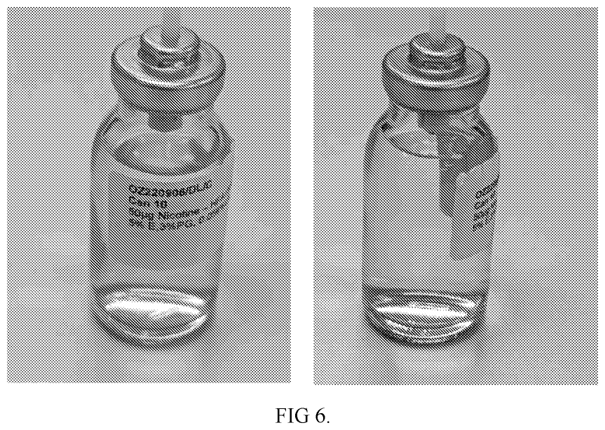

Formulations packaged in glass bottles were used to confirm the final formulations were clear solutions at T=5° C. and T=20° C. (see FIG. 6).

Formulations packaged in 19 ml Plasma treated canisters (Presspart) and 50 μl valves (Aptar) were used to characterise Nicotine delivery. All MDIs were actuated using Presspart 0.23 mm actuators (Presspart).

Tables 6 and 7 presents the formulations investigated.

TABLE 6
Nicotine formulation with HFA 152a as propellant.

| | | | Mass per 50 μL |
|---|---|---|---|
| Component | Mass % w/w | Per pMDI (mg) | (mg) |
| Nicotine | 0.1115 | 14.012 | 0.050 |
| Ethanol | 5.0000 | 628.320 | 2.244 |
| Propylene Glycol | 3.0000 | 376.992 | 1.346 |
| Menthol | 0.0000 | 0.000 | 0.000 |
| Glycerol | 0.0000 | 0.000 | 0.000 |
| Lactic Acid | 0.0562 | 7.062 | 0.025 |
| HFA 152a | 91.8323 | 11540.014 | 41.214 |
| Total | 100.0000 | 12566.400 | 44.880 |

TABLE 7
Nicotine formulation with HFO 1234ze as propellant.

| | | | Mass per 50 μL |
|---|---|---|---|
| Component | Mass % w/w | Per pMDI (mg) | (mg) |
| Nicotine | 0.0880 | 14.020 | 0.050 |
| Ethanol | 5.0000 | 796.600 | 2.845 |
| Propylene Glycol | 3.0000 | 477.960 | 1.707 |
| Menthol | 0.0000 | 0.000 | 0.000 |
| Glycerol | 0.0000 | 0.000 | 0.000 |
| Lactic Acid | 0.0562 | 8.954 | 0.032 |
| HFA 152a | 91.8558 | 14634.466 | 52.266 |
| Total | 100.0000 | 15932.000 | 56.900 |

Particle size distributions was evaluated by Anderson Cascade Impactor fitted with a USP induction port (USP Apparatus 1). Dose uniformity was determined by DUSA (USP Apparatus A). Anderson Cascade Impactors, USP induction ports and DUSA apparatus were chilled overnight at 5° C. by refrigeration.

A summary of the observed Drug Delivery Metrics is presented in Table 8.

TABLE 8
Summary of Drug Delivery Metrics

| | Batch | | | | |
|---|---|---|---|---|---|
| | OZ220824DSA & B | OZ220906C | | OZ220906DLD | |
| | (n = 4) | (n = 2) | | (n = 2) | |
| Propellant | HFA 134a | HFA 152a | | HFO1234ze | |
| Metered Dose (μg) | 51 ± 2 | 47 | 48 | 49 | 51 |
| Delivered Dose (μg) | 46 ± 3 | 41 | 43 | 44 | 46 |
| Fine Particle Dose | 16 ± 1 | 13 | 13 | 16 | 17 |
| (μg) | | | | | |
| Fine Particle | 35 ± 3 | 32 | 30 | 36 | 36 |
| Fraction (μg) | | | | | |
| MMAD (μm) | 4.5 ± 0.3 | 4.9 | 5.0 | 4.8 | 4.9 |

| | | | |
|---|---|---|---|
| Shot Weight (mg) | 61.7 ± 0.6, | 45.5 ± 0.8 | 59.7 ± 0.7 |
| | 62.8 ± 0.6 | | |

The metered dose was consistent for all formulations and close to the 50 μg/50 μl target.

Shot weights and drug delivery metrics were consistent between formulations containing HFA 134a and HFO1234ze propellants. The lower shot weight of the HFA 152a formulation is a consequence of the low density of the propellant (0.9 g/ml) relative to HFA 134a (1.226 g/ml) and HFO 1234ze (1.17 g/ml).

In conclusion, the results demonstrate that each of the three tested propellants (HFA 134a, HFA 152a and HFO1234ze) can be used to prepare MDI nicotine formulations with the desired characteristics.

1. An inhalable formulation comprising:
nicotine, or a pharmaceutically acceptable derivative or salt thereof;
at least 90% w/w of a propellant;
between 2%-8% w/w of a C₁ to C₆ alcohol; and
between 1.5%-5% w/w of a glycol.

2. The inhalable formulation of claim 1, wherein the nicotine, or a pharmaceutically acceptable derivative or salt thereof, is present at between about 200 μg/50 μL w/v to about 50 μg/50 μL w/v.

3. The inhalable formulation of claim 1, wherein the propellant is a hydrofluorocarbon propellant.

4. The inhalable formulation of claim 3 wherein the hydrofluorocarbon propellant is selected from the group consisting of 1,1,2,2-tetrafluoroethane (HFA 134a), 1,1-difluoroethane (HFA 152a), 1,1,1,2,3,3-heptafluoropropane (HFA 227) and trans-1,3,3,3-tetrafluoroprop-1-ene (HFO 1234ze).

5. The inhalable formulation of claim 1, wherein the C₁ to C₆ alcohol is selected from a C₁ to C₄ alcohol, a C₂ to C₄ alcohol, and a C₂ or C₃ alcohol.

6. The inhalable formulation of claim 1, wherein the C₁ to C₆ alcohol is ethanol.

7. The inhalable formulation of claim 1, wherein the glycol is selected from the group consisting of propylene glycol, polypropylene glycol and polyethylene glycol.

8. The inhalable formulation of claim 1, wherein the glycol is propylene glycol.

9. The inhalable formulation of claim 1, wherein the C₁ to C₆ alcohol is present at between 4%-6% w/w of the entire formulation.

10. The inhalable formulation of claim 1, wherein the glycol is present at between 2.0%-4.0% w/w of the entire formulation.

11. The inhalable formulation of claim 1, wherein the inhalable formulation further comprises glycerol.

12. A metered dose inhaler comprising an inhalable formulation, said inhalable formulation comprising:
nicotine, or a pharmaceutically acceptable derivative or salt thereof;
at least 90% w/w of a propellant;
between 2%-8% w/w of a C₁ to C₆ alcohol; and
between 1.5%-5% w/w of a glycol.

13. (canceled)

14. (canceled)

15. A method of treating a nicotine addiction in a subject including the steps of:
providing a first inhalable formulation of claim 1 to the subject;
allowing the subject to inhale the formulation,
to deliver nicotine to the subject and thereby treat the nicotine addiction.

16. The method of claim 15, wherein the method of treating a nicotine addiction in a subject further includes a step of administering a second inhalable formulation, the second inhalable formulation having a reduced nicotine content compared with the first inhalable formulation, wherein the second inhalable formulation comprises:
nicotine, or a pharmaceutically acceptable derivative or salt thereof;
at least 90% w/w of a propellant;
between 2%-8% w/w of a C₁ to C₆ alcohol; and
between 1.5%-5% w/w of a glycol.

17. (canceled)

18. The inhalable formulation of claim 1, comprising:
nicotine, or a pharmaceutically acceptable derivative or salt thereof;
at least 90% w/w of a propellant;
between 4.0%-6.0% w/w, of ethanol; and
between 2%-4% w/w of propylene glycol.

19. The inhalable formulation of claim 18, wherein the inhalable formulation further comprises glycerol.

20. (canceled)
